(19) United States
(12) Patent Application Publication
(10) Pub. No.: US 2011/0195433 A1
(43) Pub. Date: Aug. 11, 2011

(54) Methods for Screening of Opioid Receptor Neutral Antagonists and Inverse Agonists and Uses Thereof

(75) Inventors: Wolfgang Sadee, Upper Arlington, OH (US); Danxin Wang, Upper Arlington, OH (US)

(73) Assignee: THE OHIO STATE UNIVERSITY RESEARCH FOUNDATION, Columbus, OH (US)

(21) Appl. No.: 12/808,852

(22) PCT Filed: Dec. 16, 2008
(86) PCT No.: PCT/US08/86939

§ 371 Date: Jul. 9, 2010

Related U.S. Application Data
(60) Provisional application No. 61/007,958, filed on Dec. 17, 2007.

(51) Int. Cl. G01N 33/53

(52) U.S. Cl. 435/7.21

(57) ABSTRACT

Methods for screening of opioid receptor neutral antagonists and inverse agonists and uses thereof are disclosed.

### RELATED APPLICATIONS

This application claims priority to U.S. Provisional Patent Application 61/007,958 filed Dec. 17, 2007, the disclosure of which is incorporated herein by reference, in its entirety.

### GOVERNMENT SUPPORT

The invention was made with government support from the National Institute on Drug Abuse Grant DA04166. The government may have certain rights in the invention.

### FIELD OF INVENTION

The present invention relates to screening assays for compounds that modulate the interaction between opioids and other ligands and the G protein-coupled receptors GPCRs receptor.

### BACKGROUND

G protein-coupled receptors (GPCRs) have diverse physiological functions, and they are important pharmacological targets. Although a GPCR typically requires activation by an agonist, many GPCRs also display basal or spontaneous signaling activity in the absence of agonist (constitutive activity). The identification of inverse agonists that block basal signaling of a GPCR further confirmed the existence of basal activity, and it was subsequently suggested that a majority of currently known GPCR antagonists are inverse agonists (Kenakin, 2004). Antagonists with inverse agonist property potentially have distinct treatment outcomes compared with neutral antagonists, i.e., antagonists that block agonist activation but do not affect basal activity. For example, the classification of “typical” and “atypical” antipsychotics may in part be related to inverse agonism (atypical) and neutral antagonism (typical) at 5-hydroxytryptamine_{2C} receptors (Herrick-Davis et al., 2000).

Opiates, are a class of centrally acting compounds and are frequently used agents for pain control. Opiates are narcotic agonistic analgesics and are drugs derived from opium, such as morphine, codeine, and many synthetic congeners of morphine, with morphine being the most widely used derivative. Opioids are natural; and synthetic drugs with morphine-like actions and include the opiates. Opioids are narcotic agonistic analgesics which produce drug dependence of the morphine type and are subject to control under federal narcotics law because of their addicting properties. The chemical classes of opioids with morphine-like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives.

Physical dependence or drug addiction to narcotic drugs, for example, opioids, has been traditionally treated by drug withdrawal through administering an opioid antagonistic drug such as naltrexone or naloxone, withholding the opioid from the drug-dependent individual, gradually decreasing the amount of opioid taken by the individual over time, or substituting another drug, such as methadone, buprenorphine, or methadyl acetate, for the opioid to ameliorate the physical need for the opioid. When an opioid is discontinued, withdrawal symptoms appear, the character and severity of which are dependent upon such factors as the particular opioid being withdrawn, the daily dose of the opioid that is being withdrawn, the duration of use of the opioid, and the health of the drug dependent individual. The pain associated with withdrawal symptoms can be quite severe.

For example, the withdrawal of morphine, heroin, or other opioid agonists with similar durations of action from an individual dependent upon the opioid gives rise to lacrimation, rhinorrhea, yawning, and sweating 8 to 12 hours after the last dose of the opioid. As withdrawal progresses, the individual will be subject to dilated pupils, anorexia, gooseflesh, restlessness, irritability, and tremor. At the peak intensity of withdrawal, which is 48 to 72 hours for morphine and heroin, the individual suffers from increasing irritability, insomnia, marked anorexia, violent yawning, severe sneezing, lacrimation, coryza, weakness, depression, increased blood pressure and heart rate, nausea, vomiting, intestinal spasm, and diarrhea. The individual commonly experiences chills alternating with hot flushes and sweating, as well as abdominal cramps, muscle spasms and kicking movements, and pains in the bones and muscles of the back and extremities, and exhibits leukocytosis and an exaggerated respiratory response to carbon dioxide. Typically the individual does not eat or drink which, when combined with the vomiting, sweating, and diarrhea, results in weight loss, dehydration, and ketosis. The withdrawal symptoms from morphine and heroin usually disappear in 7 to 10 days, but the drug dependent individual suffers greatly during the withdrawal period.

Alternatively, if an opioid antagonistic drug is administered to the individual, such as naloxone or naltrexone, withdrawal symptoms develop within a few minutes after parenteral administration and reach peak intensity within 30 minutes, with a more severe withdrawal than from withholding the opioid. For example, naloxone is the current treatment of choice in cases of overdose. It is immediately effective but is encumbered by intense withdrawal syndrome. Naltrexone can be used, for example, in maintenance therapy, but is quite aversive, which impedes wide acceptance and efficacy. Since addiction to cocaine and alcohol have been reported to also be mediated by specific opioid-sensitive brain cell networks (See, Gardner et al., Substance Abuse 2nd Ed., pp. 70-99 (1992)) the use of opioid antagonists can be suitable for use in the treatment of alcohol and cocaine dependency. Thus, the opioid receptors can play a role in the dependency of multiple drug substances.

The use of opioid analgesics for the treatment of pain and during and/or after anesthesia can also lead to unwanted side effects, for example, respiratory depression. It is frequently necessary to titrate back or adjust the degree of analgesic/anesthesia in an individual receiving opioid pain management, for example, undergoing or recovering from a surgical procedure, due to complications associated with too high of a dose. The use of naltrexone and naloxone present undesirable side effects such as exacerbation respiratory depression when used to titrate back. Further, use of opioid analgesics for chronic pain can often be associated with constipation which can be a significant and limiting problem. There is currently no known treatment strategy to reduce the constipating effects of the opioid analgesics without blocking the analgesic effect and/or causing additional side effects (e.g., diarrhea and hyperalgesia).

Therefore, a need exists for agents which can be used in the treatment of drug dependency or in pain management to, for example, modify the anesthesia/analgesia of an opioid drug or its unwanted side effects but which have reduced aversive properties and can result in reduced withdrawal symptoms.

The opioid receptors belong to GPCR family and consist of three genes encoding μ-, δ-, and κ-opioid receptors (or MOR, DOR, and KOR, respectively). Although the basal signaling activity for DOR is readily detectable (Costa and Herz, 1989), being the first GPCR found to display basal signaling activity, this was more difficult to demonstrate for MOR, possibly due to the masking of basal MOR activity by interacting regulatory proteins, such as calmodulin (Wang et al., 1999). The inventors herein have demonstrated the presence of basal MOR signaling activity in various tissues in cell culture (Wang et al., 1994, 1999, 2000; Burford et al., 2000) and in mouse brain tissue (Wang et al., 2004), which was typically more prominent in opioid agonist-pretreated (“dependent”) tissues. This was at first an unexpected finding, because MOR is thought to desensitize during agonist pretreatment; yet, we have shown that release of calmodulin from the receptor by agonist stimulation uncovered the innate basal activity of MOR in the dependent state (Wang et al., 1999, 2000).

The inventors have further identified several inverse agonists and neutral antagonists, the latter blocking both opioid agonist and inverse agonist effects, a strong indication that the observed effects are indeed elicited by binding to MOR (Bilsky et al., 1996; Wang et al., 2001, 2004). Constitutive MOR activity was independently confirmed (Liu et al., 2001), as was the unusual regulation of constitutive activity of MOR and DOR receptors by chronic agonists pretreatment (Liu and Prather, 2001, 2002). Moreover, basal opioid activity has been implicated in appetite (Emmerson et al., 2004), morphine tolerance (Heinzen et al., 2005), and methamphetamine-induced behavioral sensitization (Chiu et al., 2006).

An important finding came from the observation that some neutral antagonists, such as naloxone and naltrexone, turned into inverse agonists after agonists pretreatment, suggesting that the receptor has been modified in the dependent state. The conversion of naloxone and naltrexone from neutral antagonist to inverse agonist seems to contribute to precipitated withdrawal symptoms in opioid addicts (Wang et al., 2004; Raehal et al., 2005; Sadee et al., 2005). This was supported by our finding that neutral antagonist 6βnaltrexol and _{D}-Phe-Cys-Tyr-D-Trp-Arg-Thr-Pen-Thr-NH₂, which remain neutral in the dependent state, precipitate less withdrawal than naloxone in morphine-dependent mice, at equipotent pharmacological doses in blocking morphine-induced antinociception (Bilsky et al., 1996; Wang et al., 2004; Raehal et al., 2005). Others also reported that basal activity of MOR was related to conditioned aversive effect of naloxone in morphine-dependent mice (Shoblock and Maidment, 2006), opioid antagonists with different inverse agonist properties have different effects in precipitating withdrawal in acute morphine-dependent mice (Walker and Sterious, 2005), and constitutive opioid receptor activation is critically involved in acute opioid withdrawal (Freye and Levy, 2005).

The variable behavior of naloxone and naltrexone in naive and opioid-pretreated cells is consistent with the notion that ligand activity can vary with cellular context, i.e., the “protean” properties of the ligands (Gbahou et al., 2003). Thus, naltrexone and naloxone are protean antagonists at MOR, whereas 6β-naltrexol is neutral under all conditions studied (Wang et al., 2001; Raehal et al., 2005). A thorough understanding of the malleable protean properties of opioid antagonists is important because of the dramatic precipitated withdrawal caused by naloxone and naltrexone, effects that may be avoided or ameliorated with a neutral antagonist such as 6β-naltrexol. Moreover, these ligands also bind to DOR and KOR, but regulation of basal activity and protean ligand properties remain unknown at these subtypes

What are lacking are tools for predicting the likelihood that a particular compound will be useful and/or whether that compound will have undesired side-effects.

Such tools would likewise enable the identification of new drugs that affect the G protein coupled receptors (GPCRs) in a subject, particularly new agents that alter the potential side-effects of opioid antagonists.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### SUMMARY

In a very broad aspect, the disclosure provides for a method for screening of opioid receptor neutral antagonists and inverse agonists, comprising: providing cells stably expressing single μ-opioid receptor (MOR), δ-opioid receptor (DOR) or κ-opioid receptor (KOR), or co-expressing MOR/DOR, MOR/KOR or DOR/KOR; selecting one or more compounds showing inhibition of ³H-diprenorphine binding at MOR membranes; selecting one or more compounds showing neutral agonist properties at three receptors; selecting one or more compounds showing neutral agonist properties at MOR; and, selecting one or more compounds substantially consistently showing neutral antagonist properties at MOR in different assays.

In certain embodiments, each compound is tested in three receptors, without or with a narcotic analgesic or receptor specific agonist pretreatment.

In certain embodiments, the narcotic analgesic is morphine, and the receptor specific agonist pretreatment is an inverse agonist.

In certain embodiments, the receptor specific agonist pretreatment is an inverse agonist comprising β-naloxone.

In certain embodiments, cells stably expressing single MOR, DOR or KOR, or co-expressing MOR/DOR, MOR/KOR or DOR/KOR are established by: transfecting opioid receptors into HEK cells, and selecting single clones; culturing one or more cloned cells, pretreating the cells with morphine or subtype specific agonists, harvesting one or more the cells; using permeabilized cells or cell membranes in a GTPγS, guanosine 5′-O-(3-thio)triphosphate (GTPγS) binding assay with the one or more compounds; and, incubating the one or more compounds with the cell membranes or permeabilized cells and ³H-diprenorphine.

In certain embodiments, the pretreating step comprises pretreating the cells with morphine or subtype specific agonists comprises DAMGO, [D-Ala2,N-Me-Phe4,Gly5-ol]-enkephalin, for MOR.

In certain embodiments, the ³⁵S-GTPyS binding assay in membranes uses an assay buffer containing Tris-HCl, KCl, EDTA, MgCl₂, GDP and ³⁵S-GTPγS.

In certain embodiments, the pretreating step comprises pretreating the cells with morphine or subtype specific agonists comprises, DPDPE, [D-Pen2,D-Pen5]-enkephalin, for DOR.

In certain embodiments, the ³⁵S-GTPγS binding assay in membranes uses an assay buffer containing Tris-HCl, KCl, EDTA, MgCl₂, GDP and ³⁵S-GTPγS.

In certain embodiments, the pretreating step comprises pretreating the cells with morphine or subtype specific agonists comprises U69593, (+)-(5α,7α,8β)-N-methyl-N-[7-(1-pyrrolidinyl)-1-oxaspiro[4.5]dec-8-yl]benzeneacetamide, for KOR.

In certain embodiments, the ³⁵S-GTPγS binding assay in membranes uses an assay buffer containing Tris-HCl, NaCl, EDTA, MgCl₂, GDP and ³⁵S-GTPγS.

In certain embodiments, in the ³⁵S-GTPγS binding assay in permeabilized cells, the one or more compounds were incubated with the permeabilized cells and ³⁵S-GTPγS.

In certain embodiments, in the assay, the cells pretreated with or without morphine or receptor specific agonists, then incubated with the one or more compounds.

In another broad aspect, there is provided herein a method for testing the effects of opioid antagonists on MOR, DOR, KOR and their hetero-dimers before or after different agonists pretreatment, comprising using one or more of the methods described herein.

In another broad aspect, there is provided herein a method for identifying potential side-effects of opioid antagonists, comprising using one or more of the methods described herein.

In another broad aspect, there is provided herein a method for selecting a drug candidate for treatment of conditions where opioid antagonists are indicated, comprising using one or more of the methods described herein.

In certain embodiments, the method includes comparing of receptor activities by classifying a test compound as an agonist, neutral antagonist, or an inverse agonist.

In another broad aspect, there is provided herein a kit comprising an assay for the screening methods described herein. In certain embodiments, the kit further includes comprises instructions for correlating the assay results with the subject's risk for having or developing an adverse withdrawal symptom. In certain embodiments, the kit further includes instructions for correlating the assay results with the subject's prognostic outcome for an adverse withdrawal symptom. In certain embodiments, the kit further includes instructions for correlating the assay results with the probability of success or failure of a particular drug treatment in the subject.

In another broad aspect, there is provided herein a method for distinguishing the effects of naloxone from those of naltrexone comprising using one or more of the methods described herein.

In another broad aspect, there is provided herein a method for demonstrating different antagonists having distinct pharmacological properties comprising using one or more of the methods described herein.

A method for determining whether agonist-pretreatment increases basal activity and/or sensitizes inverse agonist effects at MOR, DOR and/or KOR, comprising using one or more of the methods described herein.

In certain embodiments, for MOR, both potency and efficacy of BNTX, 7-benzylidenenaltrexone, increases, and naloxone and naltrexone turn into inverse agonist after agonist pretreatment.

In certain embodiments, for KOR, agonist pretreatment increases efficacy and potency of nor-BNI, nor-binaltorphimine, and GNTI, 5′-guanidinyl-17-(cyclopropylmethyl)-6,7-dehydro-4,5α-epoxy-3,14-dihydroxy-6,7-2′3′-indolomorphinan dihydrochloride, affects potency.

In certain embodiments, for DOR, a decrease in efficacy of ICI 174,864, N,N-diallyl-Tyr-Aib-Aib-Phe-Leu-OH, increases after DPDPE, [D-Pen2,D-Pen5]-enkephalin, pretreatment, an/or the potency of ICI 174,864 increases after morphine pretreatment.

In yet another broad aspect, there is provided herein a method for developing safer and more effective opioid antagonists targeting a variety of clinical needs, including long-term treatment of addiction, and opioid-induced gastrointestinal dysfunction, comprising using the one or more of the methods described herein.

### BRIEF DESCRIPTION OF THE DRAWINGS

FIG. 1: Chemical structure of 6β-naltrexamide.

FIGS. 2A-2B: Regulation of agonist and inverse agonist effects on MOR after DAMGO and morphine pretreatment in transfected HEK-MOR cells. HEK-MOR cells were pretreatment with 1 μM DAMGO or 10 μM morphine for 24 h, and then cell membranes were prepared for [35S]GTPγS binding.

FIG. 2A: Dose-response curves of DAMGO in control and DAMGO-pretreated membranes.

FIG. 2B: Dose-response curves of BNTX in control, DAMGO-, or morphine-pretreated membranes. Data are means±S.D., n=6.

FIG. 3A: Inhibition of inverse agonist effects of 1 μM BNTX by naloxone (Nal), 6β-naltrexol (6β-nal), and 6β-naltrexamide (6β-NXM) (all 10 μM) in untreated HEK-MOR cell membranes.

FIG. 3B: Inhibition of inverse agonist effects of naloxone in DAMGO-pretreated HEK-MOR membranes by 6β-naltrexol and 6β-naltrexamide. Data are means±S.D., n=6. *, P<0.05 and **, P<0.01, compared with control (C).

FIGS. 4A-4B: Regulation of agonist and inverse agonist effects on DOR after DPDPE and morphine pretreatment in transfected HEK-DOR cells. HEK-DOR cells were pretreatment with 1 μM DPDPE or 50 μM morphine for 24 h, and then cell membranes were prepared for [35S]GTPγS binding.

FIG. 4A: Dose-response curves of DPDPE in control, DPDPE-, or morphine-pretreated membranes.

FIG. 4B: Dose-response curves of ICI 174,864 in control, DPDPE-, or morphine-pretreated membranes. Data are means±S.D., n=6.

FIG. 5A: Inhibition of inverse agonist effects of 0.1 μM ICI 174,864 by Nal, NTX, 6β-nal, and 6β-NXM (all at 10 μM) in untreated HEK-DOR cell membranes.

FIG. 5B: Inhibition of inverse agonist effects of naloxone in DPDPE-pretreated HEK-DOR membranes by naltrexone, 6β-naltrexol, and 6β-naltrexamide. Data are means±S.D., n=6. *, P<0.05 and **, P<0.01, compared with control (C).

FIG. 6: Dose-response curves of nor-BNI and GNTI on basal [35S]GTP binding in HEK-KOR membranes. Data are means±S.D., n=5.

FIGS. 7A-7B: Regulation of agonist and inverse agonist effects on KOR after U-69593 and morphine pretreatment in transfected HEK-KOR cells. HEK-KOR cells were pretreatment with 1 μM U-69593 or 50 μM morphine for 24 h, and then cell membranes were prepared for [35S]GTPγS binding.

FIG. 7A: Dose-response curves of U-69593 in control, U-69593-, or morphine-pretreated membranes.

FIG. 7B: Dose-response curves of nor-BNI in control, U-69593-, or morphinepretreated membranes. Data are means±S.D., n=6.

FIG. 8A: Inhibition of inverse agonist effects of 10 nM nor-BNI by Nal, NTX, 6β-nal, and 6β-NXM (all at 10 μM) in untreated HEK-KOR cell membranes.

FIG. 8B: Inhibition of inverse agonist effects of naloxone in morphine-pretreated HEK-KOR membranes by 6β-naltrexol and inhibition of inverse agonist effects of 6β naltrexol in U-69593-pretreated HEK-KOR membranes by 6β-naltrexamide. Data are means±S.D., n=6. *, P<0.05 and **, P<0.01, compared with control (C).

FIG. 9—TABLE 1: Opioid receptor binding affinity (Ki) and antagonistic potency (Ki′) of opioid antagonists on MOR, DOR, and KOR. Receptor binding affinities were measured by competitive inhibition of 0.5 nM [3H]diprenorphine binding performed in MOR, DOR, and KOR membranes. Ki=IC50/(1+L/Kd), where L is 0.5 nM, and the Kd values for [3H]diprenorphine in MOR, DOR, and KOR are 0.39, 0.44, and 0.27 nM, respectively. For antagonistic effects, 1μM DAMGO- or 30 nM DPDPE- or 300 nM U50,488H-induced responses were measured in the presence of different concentrations of the tested compounds. Ki′=IC50/(1+L/EC50), where L is the concentration of agonist used, and the EC50 values for DAMGO, DPDPE, and U50,488H are 74, 0.68, and 8.2 nM, respectively. Data are means±S.D., n=3. N.D., not determined.

FIG. 10—TABLE 2: Effects of different opioid antagonists on basal [35S]GTPγS binding in MOR cell membranes with or without agonists pretreatment. Maximal effects (Emax) are expressed as percentage of change from basal. Positive values indicate agonistic effects, whereas negative values indicate inverse agonist effects. Data are means±S.D., n=4 to 8. N.D., not determined. * P<0.01, compared with no pretreatment. ** P<0.01, compared with zero; ANOVA with Dunnett's post test.

FIG. 11—TABLE 3: Effects of different opioid antagonists on basal [35S]GTPλS binding in DOR cell membranes with or without agonists pretreatment. Data are means±S.D., n=4 to 8. N.D., not determined. * P>0.05, compared with no pretreatment; ANOVA with Dunnett's post test. ** P>0.01, compared with no pretreatment; ANOVA with Dunnett's post test. *** P<0.01, compared with zero; ANOVA with Dunnett post test.

FIG. 12—TABLE 4: Effects of different opioid antagonists on basal [35S]GTPλS binding in KOR cell membranes with or without agonists pretreatment. Maximal effects (Emax) are expressed as percentage of change from basal. Positive values indicate agonistic effects, whereas negative values indicate inverse agonistic effects. Data are means±S.D., n=4 to 8. N.D., not determined. * P>0.05, compared with no pretreatment; ANOVA with Dunnett's post test. ** P>0.01, compared with no pretreatment; ANOVA with Dunnett's post test. *** P<0.01, compared with zero; ANOVA with Dunnett's post test.

FIG. 13—TABLE 5: Regulation of basal activity and changes in ligand pharmacological properties in MOR, DOR, and KOR after morphine and receptor-selective agonist pretreatment. BNTX, ICI 174,864, and nor-BNI were used as prototype inverse agonists for MOR, DOR, and KOR, respectively. Naloxone, naltrexone, 6β-naltrexol, and 6β-naltrexamide are neutral antagonist or partial agonist in untreated opioid receptors.

FIG. 14A: Effects of opioid antagonists on basal 35S-GTPγS binding in MOR membranes without or with DAMGO (1 μM) or morphine (10 μM) pretreatment.

FIG. 14B. 6β-naltrexol and 6□-naltrexamide inhibited the inverse agonist effect of naloxone in DAMGO pretreated MOR membranes. Data are expressed as % changes from basal, positive values indicate agonist effects, while negative values indicate inverse agonist effects. Mean±SE, n=6. *, ** Compared to basal level, P<0.05, P<0.01, ANOVA with Dunnett post test.

FIG. 15A. Effects of opioid antagonists on basal 35S-GTPγS binding in DOR membranes without or with DPDPE (1 μM) or morphine (50 μM) pretreatment.

FIG. 15B. 6β-naltrexol and 6β-naltrexamide inhibited the inverse agonist effect of naloxone in DPDPE pretreated DOR membranes. Data are expressed as % changes from basal, positive values indicate agonist effects, while negative values indicate inverse agonist effects. Mean±SE, n=6. *, ** Compared to basal level, P<0.05, P<0.01, ANOVA with Dunnett post test.

FIG. 16. Effects of opioid antagonists on basal 35S-GTPγS binding in KOR membranes without or with U69593 (1 μM) or morphine (50 μM) pretreatment. Data are expressed as % changes from basal, positive values indicate agonist effects, while negative values indicate inverse agonist effects. Mean±SE, n=6. *, ** Compared to basal level, P<0.05, P<0.01, ANOVA with Dunnett post test.

FIGS. 17A and 17B. Effects of co-administration of 6β-naltrexol or 6β-naltrexamide with naloxone on the basal 35S-GTPγS binding in KOR membranes without or with U69593 or morphine pretreatment. Data are expressed as % changes from basal, positive values indicate agonist effects, while negative values indicate inverse agonist effects. Mean±SE, n=6. *, ** Compared to basal level, P<0.05, P<0.01, ANOVA with Dunnett post test.

FIG. 18. Effects of opioid antagonists on basal 35S-GTPγS binding in permeabilized MOR cells without or with morphine (10 μM) pretreatment. Data are expressed as % changes from basal, positive values indicate agonist effects, while negative values indicate inverse agonist effects. Mean±SE, n=6. *, ** Compared to basal level, P<0.05, P<0.01, ANOVA with Dunnett post test.

FIG. 19 is flow chart showing a method for screening compounds.

### DETAILED DESCRIPTION OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

The present invention may be understood more readily by reference to the following detailed description of the embodiments of the invention and the Examples included herein. However, before the present methods, compounds and compositions are disclosed and described, it is to be understood that this invention is not limited to specific methods, specific cell types, specific host cells or specific conditions, etc., as such may, of course, vary, and the numerous modifications and variations therein will be apparent to those skilled in the art. It is also to be understood that the terminology used herein is for the purpose of describing specific embodiments only and is not intended to be limiting.

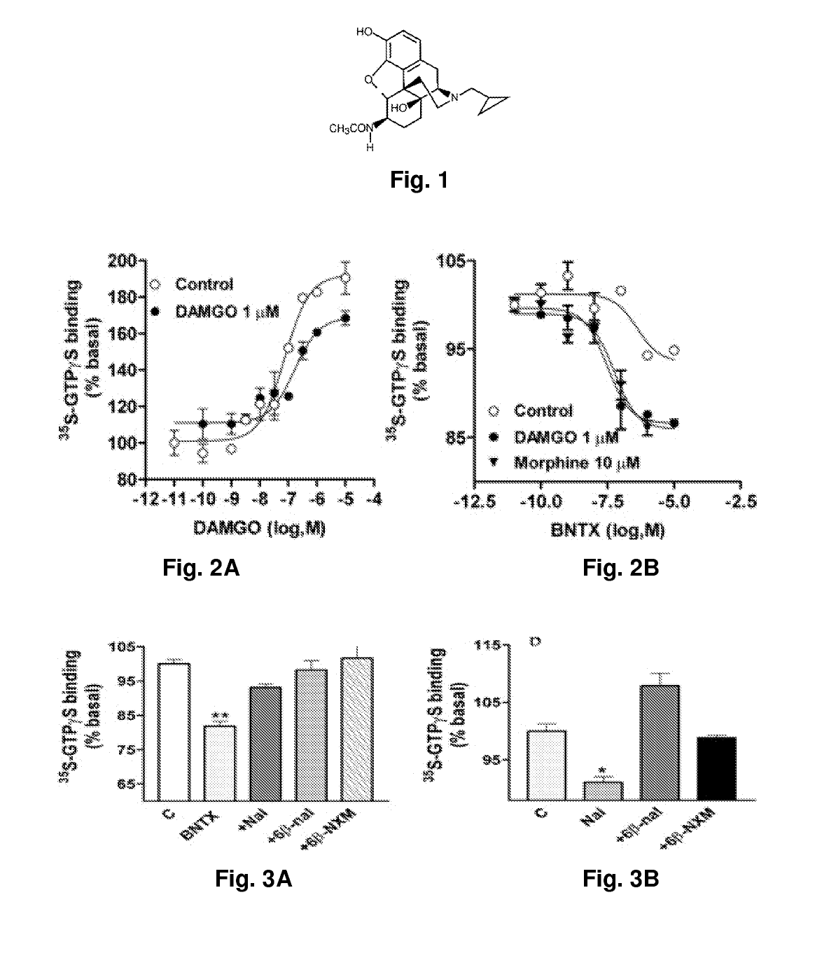

The invention herein is based, at least in part, on the inventors' discoveries resulting from their investigation and comparison of the regulation of basal activity of MOR, DOR and KOR, and the effects of naloxone, naltrexone, and naltrexone derivatives 6β naltrexol (Raehal et al., 2005) and 6β-naltrexamide (FIG. 1) on MOR, DOR, and KOR receptor with and without agonists pretreatments. We were particularly interested in 6β-naltrexol and 6β-naltrexamide, because these neutral antagonists represent potential therapeutic agents in the treatment of opioid side effect. We used BNTX (Wang et al., 2001) and ICI 174,864 (Costa and Herz, 1989) as full MOR and DOR inverse agonists, respectively. For KOR, we have identified nor-binaltorphimine (nor-BNI) and 5′-guanidinyl-17-(cyclopropylmethyl)-6,7-dehydro-4,5a-epoxy-3,14-dihydroxy-6,7-2′3′-indolomorphinan dihydrochloride (GNTI) as inverse agonist.

Materials. Morphine sulfate, naloxone, naltrexone, 6β-naltrexol, and 6β-naltrexamide were obtained through the National Institute on Drug Abuse Drug Supply Program; U-69593, [D-Pen2,D-Pen5]-enkephalin (DPDPE), and [D-Ala2,N-Me-Phe4, Gly5-ol]-enkephalin (DAMGO) were purchased from Sigma-Aldrich (St. Louis, Mo.); U50,488H, ICI 174,864, nor-BNI, and GNTI were purchased from Tocris Cookson Inc. (Ellisville, Mo.); [35S]GTPγS and [3H]diprenorphine were obtained from PerkinElmer Life and Analytical Sciences (Boston, Mass.). Other reagents for cell culture were from Sigma-Aldrich or Fisher Scientific (Pittsburgh, Pa.).

Cell Culture and Treatment. Human embryonic kidney (HEK) 293 cells stably transfected with human MOR (HEK-MOR), mouse DOR (HEK-DOR), and human KOR (HEK-KOR) were maintained in Dulbecco's modified Eagle's medium H16/F-12 supplemented with 10% fetal bovine serum, 100 μU/ml penicillin, 100 μg/ml streptomycin, and 200 μg/ml Geneticin (G-418; Invitrogen, Carlsbad, Calif.). The receptor expression levels were 1.2, 3.4, and 2.7 pmol/mg protein for HEK-MOR, HEK-DOR, and HEK-KOR, respectively (measured by [3H]diprenorphine saturation binding assays in cell membranes). For agonist pretreatment, 80% confluent cells were cultured in the presence MOR-, DOR-, or KOR-specific agonists DAMGO (1 DPDPE (1 or 10 U50,488H (1 or U-69593 (1 or nonspecific agonist morphine (10 or 50 μM) for 24 h before harvest. Cells were then washed thoroughly with phosphate-buffered saline (PBS) to remove the treated drugs before membrane preparations.

[35S]GTPγS Binding. Membrane preparation and [35S]GTPγS binding assays were carried out as described previously (Wang et al., 2000). In brief, cells were harvested and washed with PBS, and then the cells were homogenized in buffer containing 10 mM Tris-HCl, pH 7.4, and 0.1 mM EDTA and centrifuged at 30,000 g for 10 min. The pellets were resuspended in the same buffer and centrifuged again. The pellets from the second centrifugation were resuspended, aliquoted, and stored at −70° C. [35S]GTPS binding assays were performed using different conditions. For agonist effects, cell membranes (10 μg of protein) were incubated with drugs in 100 μl of assay buffer containing 50 mM Tris-HCl, pH 7.5, 1 mM EDTA, 100 mM NaCl, 10 mM MgCl2, and 10 μM GDP at 30° C. for 5 min. For inverse agonist effects, cell membranes (50 μg of protein) were incubated with drugs in 500 μl of different assay buffer containing 50 mM Tris-HCl, pH 7.5, 1 mM EDTA, and 10 μM GDP and 150 mM NaCl or KCl and different concentrations of MgCl2 (for MOR, 150 mM KCl, 1 mM MgCl2; for DOR, 150 mM KCl and 10 mM MgCl2; and for KOR, 150 NaCl and 10 mM MgCl2). The mixtures were incubated at 30° C. for 30 min. After incubation, the reactions were stopped by adding 500 μl of ice-cold PBS followed by centrifugation at 13,000 g for 10 min at 4° C. The pellets were washed once with 1 ml of PBS, and radioactivity was measured by liquid scintillation counter.

[3H]Diprenorphine Binding. For [3H]diprenorphine saturation binding assay, membranes (20 μg of protein) were incubated with different concentrations (0.5-5 nM) of [3H]diprenorphine in buffer containing 50 mM Tris-HCl, pH 7.4, and 5 mM EDTA at 23° C. for 1 h. For competitive binding experiments, 0.5 nM [3H]diprenorphine was incubated with 20 μg of membranes in the absence or presence of different concentration of tested compounds at 23° C. for 1 h. Incubations were terminated by rapid filtration onto glass fiber filters (Whatman Schleicher and Schuell, Keene, N.H.). The filters were washed with 10 ml of ice-cold PBS, and the radioactivity was quantified using a liquid scintillation counter.

Data Analysis. Results are expressed as means±S.D. for at least three experiments, each performed in duplicate. Statistical analysis and curve fits of dose-response curves were performed using Prism (GraphPad Software Inc., San Diego, Calif.).

### Results

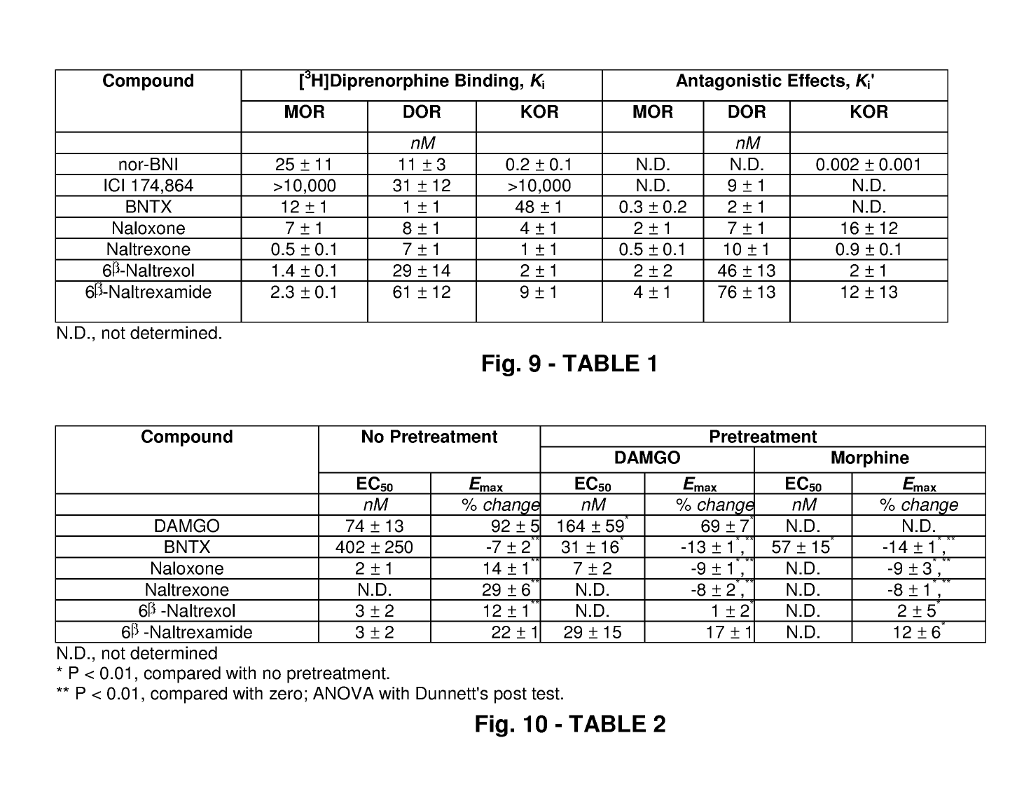

Binding Characteristics and Antagonistic Effects of Naltrexone Analogs on MOR, DOR, and KOR. The inventors performed competitive receptor binding assays in membrane expressing MOR, DOR, and KOR to determine the binding affinity of test compounds, using the tracer [3H]diprenorphine, which is a nonselective opioid ligand. Saturation binding assays showed that the Kd values of [3H]diprenorphine were 0.39, 0.44, and 0.27 nM in MOR, DOR, and KOR membranes, respectively. Both 6β-naltrexol and 6β-naltrexamide have highest affinity for MOR, followed by KOR, whereas they are 20 to 30 times less potent for DOR (FIG. 9-Table 1).

The binding affinity of 6β-naltrexol for MOR and KOR is 2- to 5-fold higher than naloxone and 2-fold less potent than naltrexone, whereas it is 3- to 4-fold less potent than naloxone and naltrexone for DOR (FIG. 9—Table 1). The binding affinity of 6β-naltrexamide is 3-fold more potent than naloxone and 4-fold less potent than naltrexone at MOR, whereas it is 2- to 7-fold and 7- to 10-fold less potent than naloxone at KOR and DOR, respectively (FIG. 9—Table 1).

To test the antagonistic properties of these compounds, the inhibition of submaximal concentration of DAMGO-(1 DPDPE-(30 nM), or U50,488H-(300 nM) stimulated [35S]GTPγS binding was determined in MOR, DOR, and KOR membranes. Naloxone, naltrexone, and analogs dose-dependently inhibited agonist-stimulated [35S]GTPγS binding in MOR, DOR, and KOR membranes with similar potency order as observed for binding affinity (FIG. 9—Table 1). This result indicates that naloxone, naltrexone, 6β-naltrexol, and 6β-naltrexamide all act as full antagonists at each of the three opioid receptors tested (Wang et al., 2001). On the other hand, the antagonistic potency (Ki′) of BNTX, ICI 174,864, and nor-BNI is greater than the [3H]diprenorphine binding affinities (Ki) (FIG. 9—Table 1). This result may be related to their status as full inverse agonists at the respective opioid receptors (see below).

Basal Signaling Activity of MOR and the Effects of Naltrexone Analogs. Pretreatment of MOR with morphine increases the inverse agonist effect of β-chloronaltrexamine (Wang et al., 2000, 2001). To test the effects of DAMGO pretreatment on basal activity and inverse agonist effects, we pretreated MOR with DAMGO for 24 h, followed by removal of the agonist by washing the cells thoroughly. DAMGO pretreatment increased the EC50 value of DAMGO and decreased the Emax value (FIG. 2A; FIG. 10—Table 2), indicating receptor desensitization and down-regulation has occurred. In contrast, both DAMGO and morphine pretreatment increased inverse agonist effects of BNTX and shifted the dose-response curve of BNTX to the left, with no difference observed between morphine and the receptor-selective agonist DAMGO (FIG. 2B; FIG. 10—Table 2).

These results demonstrate that morphine and DAMGO pretreatment increases the inverse agonist effects of BNTX, implying increased basal signal activity of MOR, consistent with previous results (Wang et al., 2001), and also sensitized MOR to the inverse agonist effects of BNTX, even though the receptor was partially desensitized to agonist activation. Similar to previous results (Wang et al., 2001), naloxone and naltrexone marginally increased basal [35S]GTPγS binding in untreated MOR membranes, but decreased it after DAMGO or morphine pretreatment with similar EC50 values (FIG. 10—Table 2). This is consistent with previous observations indicating that morphine or DAMGO pretreatment turns naloxone and naltrexone into inverse agonists. In contrast, 6β-naltrexol and 6β-naltrexamide did not decrease basal [35S]GTPγS binding even in agonist-pretreated MOR membranes (FIG. 10—Table 2), consistent with neutral antagonism without protean property as reported previously for 6β-naltrexol (Wang et al., 2001).

As a critical test of basal MOR activity, neutral antagonists are expected to inhibit the effects of inverse agonist. The inverse agonist effect of BNTX was inhibited by naloxone and naltrexone analogs in untreated MOR membranes, and the inverse agonist effect of naloxone in DAMGO-pretreated MOR membranes was also inhibited by the naltrexone analogs (FIGS. 3A and 3B). These results indicate that 6β-naltrexol and 6β-naltrexamide are neutral antagonists regardless of agonist pretreatment. Although part of these results have been reported previously (except for 6β-naltrexamide), they are essential in this report for direct comparison with subsequent results obtained under identical conditions with DOR and KOR.

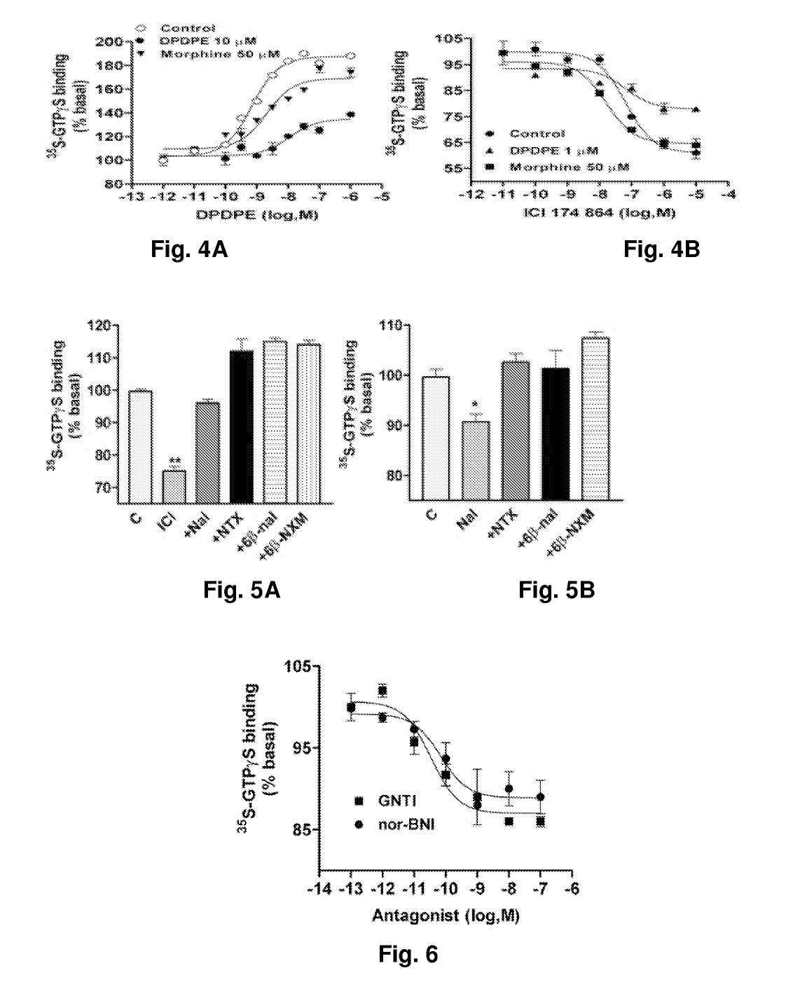

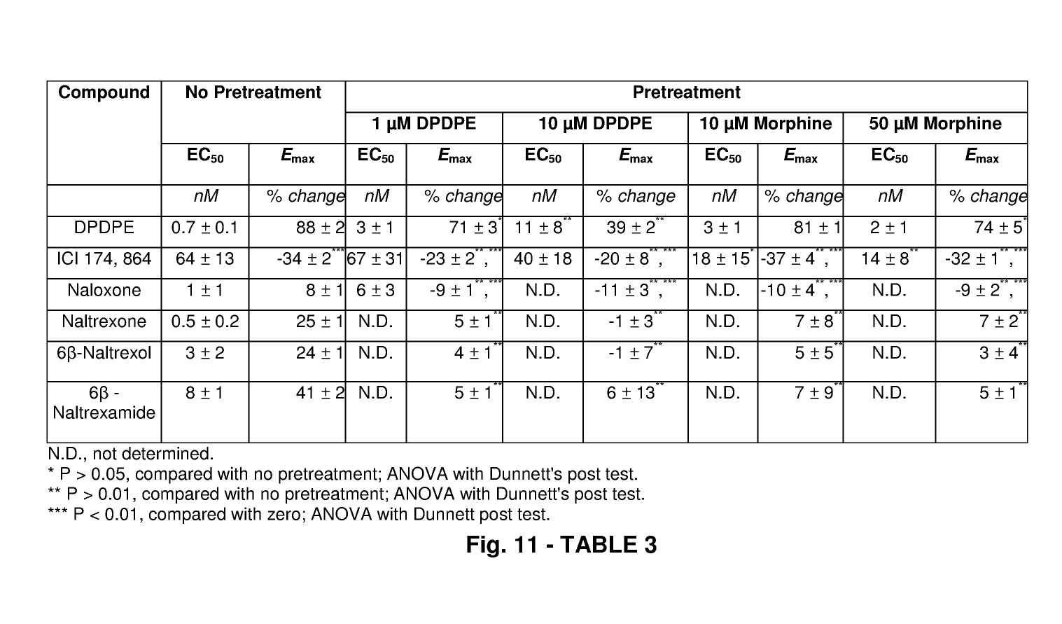

Basal Signaling Activity of DOR and the Effects of Naltrexone Analogs. Pretreatment of DOR with DPDPE has been shown to decrease the inverse agonist effect of ICI 174,864 while turning naloxone into an inverse agonist (Liu and Prather, 2002). To test whether DPDPE and morphine exposure regulates DOR basal activity differently, we pretreated HEK-DOR cells with DPDPE or morphine for 24 h, followed by washout of the agonists. Agonist pretreatment increased the EC50 value and decreased the Emax value for DPDPE (FIG. 4A; FIG. 11—Table 3), indicating receptor desensitization and down-regulation. Different from MOR, and consistent with previous results, pre-exposure of DOR to DPDPE decreased inverse agonist effects of ICI 174,864 without changing the EC50 value (FIG. 4B; FIG. 11—Table 3). In contrast, pretreatment of DOR with morphine shifted the dose-response curve for ICI 174,864 to the left without affecting Emax (FIG. 4b; FIG. 11—Table 3).

These results show that DPDPE and morphine regulate basal DOR activity differently. Similar to MOR, in untreated DOR membranes, naltrexone and its analogs showed a small increase (8-41%) in [35S]GTPγS binding, indicating partial agonist activity in this membrane preparation (FIG. 11—Table 3). In DPDPE-pretreated membranes, naloxone decreased basal [35S]GTPγS binding, indicating conversion into an inverse agonist as reported previously (Liu and Prather, 2002). Likewise, morphine pretreatment also converted naloxone into inverse agonist (FIG. 11—Table 3). However, in contrast to the findings with MOR, naltrexone did not turn into inverse agonist at DOR after either DPDPE or morphine pretreatment (FIG. 11—Table 3). On the other hand, 6β-naltrexol and 6β-naltrexamide remained neutral after both agonists pretreatment (FIG. 11—Table 3); therefore, they are neutral antagonists regardless of agonist pretreatment in DOR, as shown for MOR. The inverse agonist effect of ICI 174,864 in untreated DOR membranes was inhibited by all four antagonists, and the inverse agonist effect of naloxone in DPDPE-pretreated membranes was inhibited by naltrexone and analogs (FIG. 5). These results for the first time distinguish the effects of naloxone from those of naltrexone.

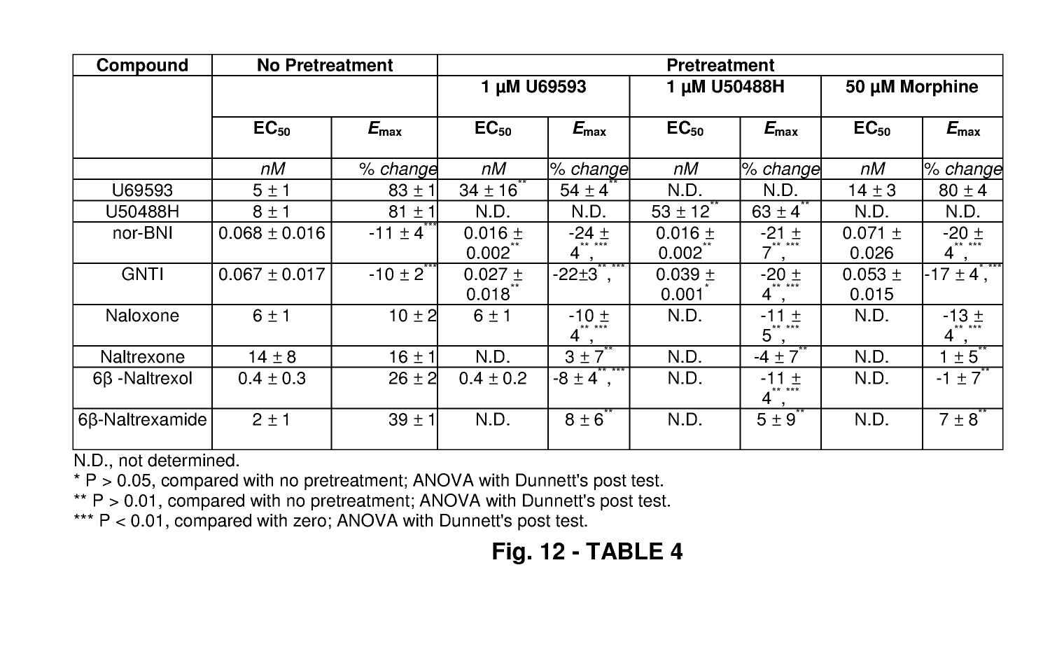

Basal Signaling Activity of KOR and the Effects of Naltrexone Analogs. Different from MOR and DOR, basal activity for KOR had not been fully demonstrated, although one KOR antagonist was found to have inverse agonist effect on KOR in a ligand screen (Becker et al., 1999. To test whether KOR displays basal activity, we tested the effects of KOR antagonists nor-BNI and GNTI on basal [35S]GTPγS binding in KOR membranes. As shown in FIG. 6, nor-BNI and GNTI dose-dependently decreased basal GTPγS binding with EC50 values in the femtomolar range and an Emax value of ˜10% FIG. 12—Table 4). These results support the existence of basal activity of KOR and indicate that nor-BNI and GNTI are inverse agonists at KOR.

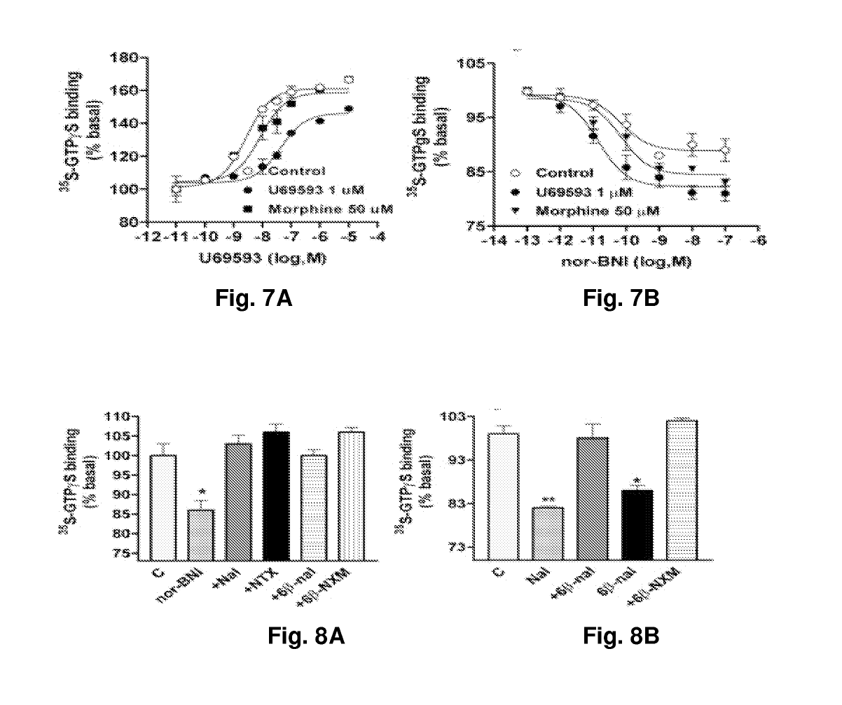

To test whether agonist pretreatment changes basal activity and/or inverse agonist effects in KOR, we pretreated HEK-KOR with the KOR-selective agonist U-69593, followed by washout. Pretreatment of KOR with 1 μM U-69593 shifted the dose-response curve of U-69593 to the right and decreased Emax (FIG. 7A; FIG. 12—Table 4), indicating KOR desensitization and down-regulation. As observed for MOR, U-69593 pretreatment of KOR increased the inverse agonist effects of nor-BNI and GNTI, and additionally, decreased their EC50 value 3- to 4-fold (FIG. 7A; FIG. 12—Table 4). Similar results were obtained by pretreatment with another KOR-selective agonist U50,488H (FIG. 12—Table 4). Morphine also stimulated KOR with an EC50 value of 185 nM and an Emax value similar to that of U-69593, in this membrane preparation. Pretreatment of KOR with morphine (10 or 50 μM) also increased the EC50 value for U-69593, without affecting the Emax value (FIG. 12—Table 4). Moreover, morphine pretreatment increased the inverse agonist effects of nor-BNI and GNTI, but in contrast to U-69593 or U50,488H pretreatment, it did not alter the EC50 values (FIG. 12—Table 4). These results show that U-69593/U50,488H and morphine pretreatment regulate basal KOR activity differently.

To test whether the naltrexone analogs have inverse agonist effects at KOR, we measured their effects on [35S]GTPγS binding in untreated KOR membranes. As with MOR, all four compounds showed no effect or small partial agonist effects, with Emax values ranging from 10 to 40% (FIG. 12—Table 4). We then tested the effects of these compounds on agonist-pretreated KOR membranes. Naloxone decreased basal [35S]GTPγ S binding in U-69593, U50,488H, and morphine-pretreated KOR membranes (FIG. 12—Table 4), as reported for MOR. In contrast to MOR, however, 6β-naltrexol decreased basal [35S]GTPγS binding in U-69593- and U50,488H-, but not morphine-pretreated KOR membranes (FIG. 12—Table 4). Naltrexone and 6β-naltremaxide remained neutral regardless of agonist pretreatment. These results demonstrate that different antagonists have distinct pharmacological properties at the three opioid receptors after different agonist pretreatments. Dose-response curves showed that the EC50 values were similar for 6β-naltrexol, acting as partial agonist or inverse agonists in untreated compared with pretreated membranes, indicating that the same binding sites are involved (FIG. 12—Table 4).

Naloxone, naltrexone, and its two analogs were able to inhibit the inverse agonist effects of nor-BNI in control KOR membranes (FIG. 8). Moreover, 6β-naltrexol inhibited inverse agonist effect of naloxone in morphine-pretreated KOR membranes (FIG. 8). Likewise, 6-naltrexamide inhibited inverse agonist effect of 6β-naltrexol in U-69593-pretreated membranes (FIG. 8).

### Discussion

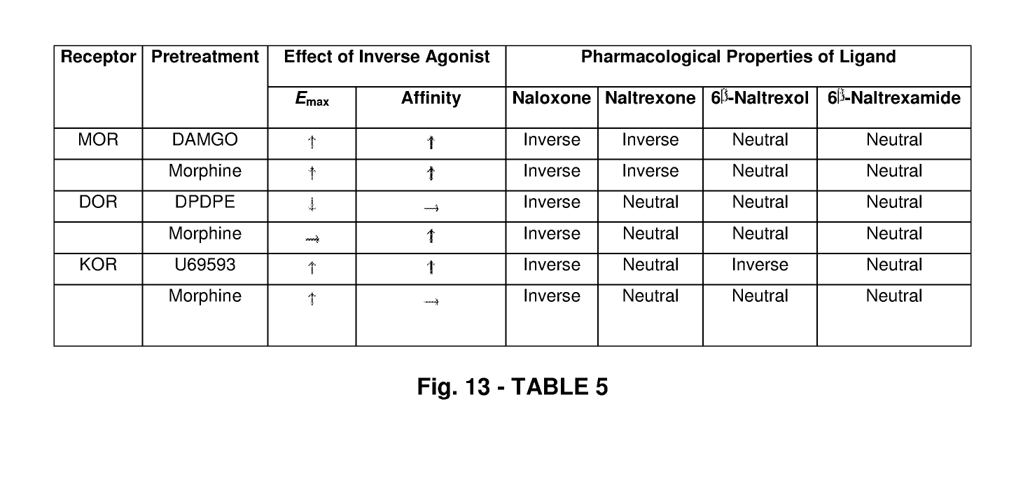

The inventors now show herein that each of the three opioid receptors displayed basal activity in transfected HEK cell membranes, which was modulated by agonist pretreatment. However, the regulation of basal signaling differs in some detail for MOR, DOR, and KOR, and with pretreatment by different agonists (FIG. 13—Table 5). These results now show that basal receptor activity can have physiological significance, and moreover, that it is subject to regulation as a result of receptor stimulation.

In contrast to agonist-induced receptor desensitization, these results indicate that agonist-pretreatment increases basal activity and/or sensitizes inverse agonist effects at MOR, DOR, and KOR, based on the following observations (FIG. 13—Table 5):

1) For MOR, both potency and efficacy of BNTX increased, and naloxone and naltrexone turned into inverse agonist after agonist pretreatment.

2) For KOR, agonist pretreatment increased efficacy and potency of nor-BNI and GNTI (with the exception of morphine, which did not affect potency). In addition, naloxone and 6β-naltrexol turned into inverse agonist after agonist pretreatment.

3) For DOR, although we observed a decrease in efficacy of ICI 174,864 after DPDPE pretreatment (possibly a result of partial receptor down-regulation), the potency of ICI 174,864 was increased after morphine pretreatment, indicating sensitization to inverse agonism after morphine pretreatment. Moreover, both morphine and DPDPE pretreatments turned naloxone into an inverse agonist.

The inventors herein note that it seems paradoxical that basal activity and inverse agonist properties of antagonists tend to increase after agonist pretreatment, whereas agonist-stimulation is desensitized. One possibility is the unmasking of basal receptor activity, for example by shedding calmodulin from binding sites in the i3 loop involved in G protein coupling (Wang et al., 1999). Increased constitutive state of receptor probably is associated with a change in receptor conformation after agonist pretreatment. This is supported by opioid receptor mutants that have enhanced constitutive activity (Brillet et al., 2003). The different pharmacological behavior of opioid antagonists may be caused by different affinities for various receptor conformations they act on. Previous studies have shown that pretreatment with the inverse DOR agonist (ICI 174,864) produces new receptor sites with 1000-fold higher affinity for naloxone at DOR (Pineyro et al., 2005). Alternatively, basal signaling could proceed via distinct signaling pathways that are activated while agonist-stimulated pathways are down-regulated. Switching of signaling pathways has been reported between different ligands and after pretreatment (Chakrabarti et al., 2001; Dupre et al., 2004). In either case, agonist pretreatment alters the opioid receptor system, modulating both agonist-stimulated and basal signaling.

The inventors' discoveries are consistent with their earlier discoveries (Wang et al., 2000, 2001, 2004) as well as a report by Liu and Prather (2001), showing that the effect of the inverse MOR agonist β-chloronaltrexamine was increased after morphine pretreatment. The results described herein did not show differences in inverse agonist effects between morphine and DAMGO pretreatment, inconsistent with a previous report showing DAMGO pretreatment increased inverse agonist effects more strongly than morphine (Liu and Prather, 2001). The discrepancy may be caused by the different cell culture (GH3 cell in the previous study) or by different DAMGO concentrations used for pretreatment. In the present example, we have chosen concentration of agonists based on their ability to produce similar response (1 μM for DAMGO and 10 μM for morphine), whereas the previous report used the same dose (10 μM for both). Because DAMGO is known to be more efficacious than morphine, the inventors now believe it to have greater effects on receptor regulation at equal doses. However, whereas agonist-stimulated responses are more strongly affected by DAMGO than morphine, this was not the case for the regulation of basal activity in this case. The inventors herein now propose that different mechanisms play a role in these two processes, an interesting possibility, because it then might be feasible to develop ligands that have differential effects and improved in vivo pharmacology. For DOR, our results are consistent with those of others (Liu and Prather, 2002), showing the effect of ICI 174,864 decreased after DPDPE pretreatment, whereas naloxone turned into an inverse agonist. The decrease in the Emax value of ICI 174,864 might have been caused by substantial receptor internalization and down-regulation after DPDPE pretreatment, which does not occur after morphine pretreatment. It is also possible that internalized receptor may not be available to the hydrophilic peptide ligand ICI 174,864. This can be further analyzed using a nonpeptide inverse agonist of DOR, such as RTI compounds (Zaki et al., 2001). The emergence of inverse agonist properties of naloxone after DPDPE indicates that the receptor is more sensitive to the antagonist, as observed with MOR. The increased potency of ICI 174,864 after morphine pretreatment also supports a process of sensitization of inverse agonism after morphine pretreatment of DOR, similar to that of MOR (calmodulin is likely to bind to DOR as well, having an identical i3 loop).

In interpreting these examples, one needs to consider that transfected cell lines and membrane preparations may not reflect the true pharmacological properties encountered in vivo. Nevertheless, we have taken the results from such in vitro studies, and similar data obtained with mouse brain membrane preparations, to predict pharmacological properties in vivo. Specifically, antagonists found to be neutral even after agonist pretreatment of MOR were subsequently shown to cause significantly less withdrawal in morphine-dependent mice (Bilsky et al., 1996; Wang et al., 2001, 2004; Raehal et al., 2005). No such in vitro-in vivo correlations exist for DOR and KOR, but the present in vitro data can form a foundation for testing different opioid antagonist properties in vivo. One further needs to consider that the cell lines are expressing high levels of opioid receptors, and the membrane incubations are done under conditions that facilitate measurements of basal activity. Under these conditions, even antagonists considered devoid of agonist activity did show some stimulation of G protein coupling, as has been observed previously (Wang et al., 2001, 2004.). Last, the magnitude of the measured inverse effects is typically less than agonist-stimulated effects. For the latter, one chooses conditions that minimize basal GTP binding by adding high GDP concentrations, whereas one lowers GDP levels to observe basal coupling. Yet, this increases background noise and hence reduces the percentage of decrease that can be observed for inverse agonists. The present in vivo data indicate that the basal signaling levels and inverse effects observed for MOR are relevant to measured antagonist effects in morphine-dependent mice (Wang et al., 2004). Similar relationships may hold for DOR and KOR.

These varying antagonist properties can be translated into differential pharmacological properties in vivo, in particular in eliciting withdrawal, both centrally and in the peripheral nervous system.

The examples herein also demonstrates qualitative differences between naloxone and naltrexone, both thought to represent prototypical opioid antagonists. Conversion of these two antagonists into inverse agonists at MOR is thought to underlie at least in part their potent ability to precipitate withdrawal in an opioid-dependent state (Wang et al., 2004). However, only naloxone converted into an inverse agonist at DOR and KOR, whereas naltrexone did not. Upon titrating naloxone and naltrexone dose-response curves in measuring various withdrawal effects, clear differences emerge at higher dose levels. The results herein are useful for developing safer and more effective opioid antagonists targeting a variety of clinical needs, including long-term treatment of addiction, and opioid-induced gastrointestinal dysfunction.

The opioid antagonist naltrexone has been used to treat opioid overdose, opioid addiction (Gonzalez et al., 2004), and addictions to other drugs of abuse, such as alcohol (Davidson et al., 1999; Chick et al., 2000). Aversive effects of naltrexone, which is similar to opioid withdrawal and occurs even in patients without pre-exposure to opioids (Hollister et al., 1981), limit its widespread use. Neutral opioid antagonists, such as 6β-naltrexol, are useful for causing less aversive effects in opioid-dependent subjects. As a metabolite of naltrexone in humans, but not in rodents, 6β-naltrexol has been suggested to contribute to the long-term duration of naltrexone action in human (Cone et al., 1974; Verebey et al., 1976.

Moreover, serum 6β-naltrexol levels are related to alcohol responses in heavy drinkers after naltrexone administration (McCaul et al., 2000), and 6β-naltrexol also reduces alcohol consumption in rats (Rukstalis et al., 2000; Stromberg et al., 2002). Although MOR is the main target receptor in narcotic analgesia and dependence, DOR and KOR also contribute to these processes, either through heterodimerization with MOR or through presynaptic/postsynaptic regulation of MOR (Narita et al., 2001; Khotib et al., 2004; Wang et al., 2005).

### EXAMPLES

The invention may be better understood by reference to the following examples, which serve to illustrate but not to limit the present invention.

### Procedures:

Cell culture and treatment: HEK cells stably expressing single MOR, DOR or KOR, or co-expressing MOR/DOR, MOR/KOR or DOR/KOR were established by transfection of opioid receptors into HEK cells and selection of single clones. Cells were cultured in DMEM/F12, supplemented with 10% fetal bovine serum and antibiotics. Cells were pretreated with morphine or subtype specific agonists (DAMGO for MOR, DPDPE for DOR and U69593 for KOR) for 24 hrs, and then harvested.

Membrane preparations: After pretreatment, cells were washed 4 times with phosphate buffer saline, the cells were homogenized in buffer containing 10 mM Tris-HCl, 0.1 mM EDTA. The homogenates were centrifuged at 800 g for 5 min. The pellets were discarded and the supernatants were centrifuged at 27,000 g for 15 min. The pellets were suspended in the same buffer and centrifuged again. The pellets from the second centrifugation were suspended in 0.5 ml in the same buffer, aliquot and stored at −70° C. until use.

Cell permeabilization: After pretreatment, cells were washed 4 times with phosphate buffer saline and suspended in the assay buffer. Cells were permeabilized with 20 μg/ml saponin in assay buffer for 5 min at 25° C. After washed twice with assay buffer, the cells are ready for GTPyS binding assay.

³H-diprenorphine binding assay: Compounds were incubated with 10 μg cell membranes or permeabilized cells and 0.5 nM ³H-diprenorphine in buffer containing 50 mM Tris-HCl, 10 mM EDTA in a total volume of 0.5 ml for 1 hr at 25° C. The reactions were stopped by rapid filtration through glass-fiber filter. The radio-activities on the filters were measured with scintillation counter.

³⁵S-GTPγS binding assay in membranes: Assay buffers are different for different receptors. For MOR and DOR, the assay buffer contains 50 mM Tris-HCl, pH 7.5, 150 mM KCl, 1 mM EDTA, 10 mM MgCl₂, 1 μM GDP and 0.25 nM ³⁵S-GTPγS. For KOR, we used 100 mM NaCl instead of KC1. The compounds (10 μM) were incubated with 20 μg membranes, 0.25 nM ³⁵S-GTPγS in 200 μl volume of assay buffer at 30° C. for 30 min. The reaction were stopped by adding 0.5 ml ice-cold phosphate buffer saline and centrifuged at 27,000 g for 5 min. The pellets were washed once with 1 ml ice-cold phosphate buffer saline and the ³⁵S-radio-activities were measured with scintillation counter.

³⁵S-GTPγS binding assay in permeabilized cells: The compounds were incubated with ˜25,000 cells and 0.5 nM ³⁵S-GTPγS in 200 μl assay buffer for 30 min at 30° C., then the reactions were stopped by adding 0.5 ml ice-cold phosphate buffer saline and centrifuged at 12,000 g for 3 min. After washing once with 1 ml phosphate buffer saline, the radio-activities were measured with scintillation counter.

cAMP assay: Cells were cultured in 24 well-plates, pretreated with or without morphine or receptor specific agonist for 24 hrs. Cells were washed once with culture medium and 3 times with serum-free medium. Then cells were incubated with compounds (10 μM) in 0.5 ml serum-free medium containing 10 μM forskolin at 37° C. for 30 min. The reactions were stopped by removing the medium and adding 0.25 ml acidify ethanol. The cAMP contents were measured by ³H-cAMP assay kit (Amersham).

Experiment #1: Initial assessment of receptor binding affinity using ³H-diprenorphine binding assay. Only one concentration of compounds was used.

Compounds showing 100% inhibition of ³H-diprenorphine binding at MOR membranes were selected.

Experiment #2: Effects of compounds (10 μM) on basal receptor-G protein coupling in control MOR, DOR and KOR membranes using ³⁵S-GTPγS binding assay.

Compounds showing neutral agonist properties at three receptors were selected.

Experiment #3: Effects of compounds (10 μM) on basal receptor-G protein coupling in morphine or receptor specific agonist pretreated MOR, DOR or KOR membranes, using ³⁵S-GTPγS binding assay.

Compounds showing neutral agonist properties at MOR were selected.

Experiment #4: cAMP assay was performed.

Experiment #5: ³⁵S-GTPγS binding in permeabilized cells. Control cells or morphine/receptor specific agonist pretreated MOR, DOR and KOR singly expressing cells, as well as MOR/DOR MOR/KOR or DOR/KOR co-expressing cells were used.

Compounds consistently showing neutral antagonist properties at MOR in different assays were selected for further analysis, by for example, dose-response curves, binding Ki, antagonistic effect, etc.

In a general aspect, each compound was tested in three receptors, without or with morphine or receptor specific agonist pretreatment. Compounds showing neutral antagonist property at MOR under any conditions were selected for further testing. The steps are shown in FIG. 6.

Using the procedure shown in FIG. 6, naltrexone analogues, 6β-naltrexol and 6β-naltrexamide, were identified as MOR neutral antagonists. Compared to naloxone and naltrexone, 6β-naltrexol and 6β-naltrexamide show similar receptor binding affinity and antagonistic potency at MOR and KOR, but relative lower affinity at DOR (as shown in FIG. 9—Table 1).

FIG. 9—Table 1, shows the opioid receptor binding affinity (Ki) and antagonistic potency (Ki′) of opioid antagonists on MOR, DOR and KOR. The receptor binding affinities were measured by competitive inhibition of ³H-diprenorphine binding (0.5 nM) performed in MOR, DOR and KOR membranes. Ki=IC₅₀/(1+L/Kd), where Kd=0.27 nM, L=0.5 nM. For antagonistic effects, 1 μM DAMGO or 30 nM DPDPE or 300 nM U50488H induced response were measured in the presence of different concentrations of the tested compounds. Ki′=IC₅₀/(1+L/EC50), where L is the concentration of agonist used, EC₅₀ for DAMGO, DPDPE and U50488H were 74, 0.68 and 8.2 nM, respectively. Mean±SD, n=3, n.d, not determined.

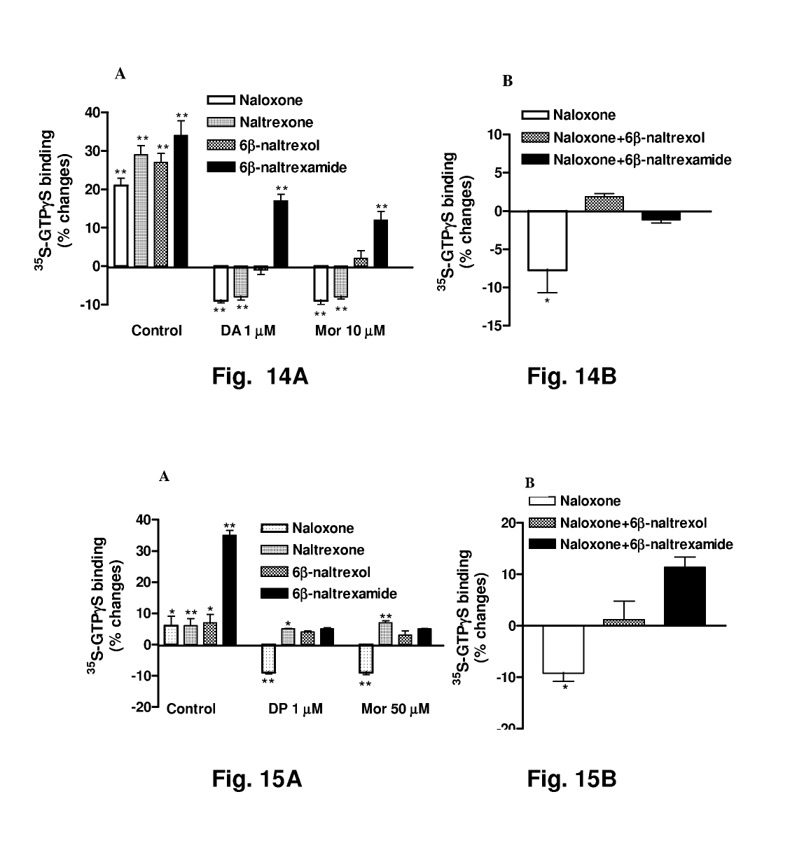

In naïve MOR membranes, 6β-naltrexol and 6β-naltrexamide showed weak agonistic effects, increasing ³⁵S-GTPγS binding ˜10-40% over the basal, similar as naloxone and naltrexone. After morphine or DAMGO pretreatment, naloxone and naltrexone turned into inverse agonists, with decreased GTPyS binding ˜10%. In contrast, 6β-naltexol and 6β-nalrexamide had no inverse agonist effects in pretreated membranes (as shown in FIG. 14A).

Instead, 6β-naltexol and 6β-nalrexamide inhibited the inverse agonist effects elicited by naloxone in agonist pretreated MOR membranes (as shown in FIG. 14B), indicating that 6β-naltrexol and 6β-naltrexamide are neutral antagonists at MOR even after agonist pretreatment.

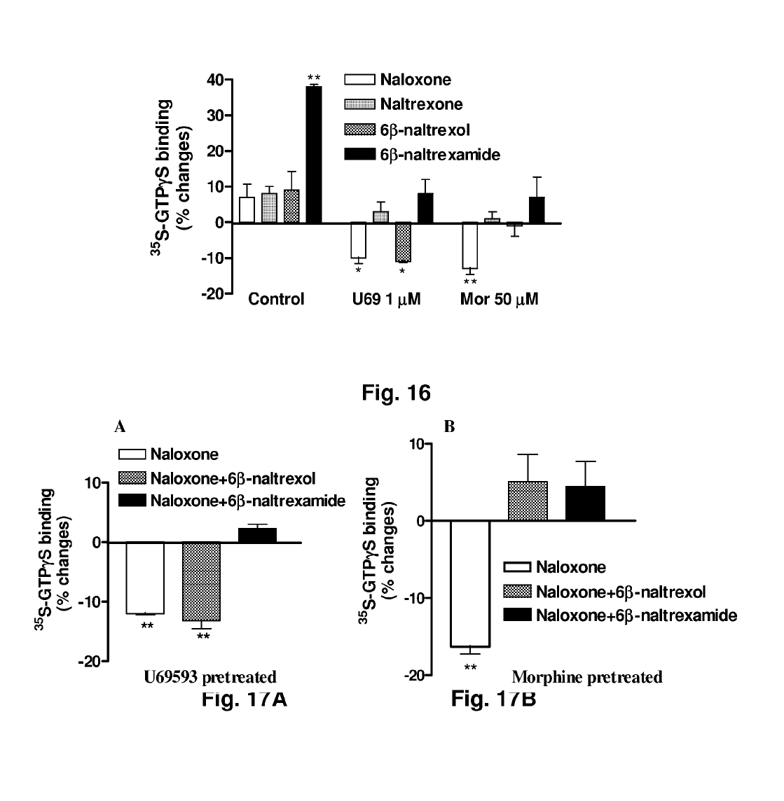

Similarly, in DOR membranes, 6β-naltrexol and 6β-naltrexamide also showed neutral antagonist property and inhibited the inverse agonist effects of naloxone in pretreated membranes (as shown in FIGS. 15A-15B).

In morphine pretreated KOR membranes, both 6β-naltrexol and 6β-naltrexamide had no inverse agonist effects by themselves and inhibited the inverse effects produced by naloxone (as shown in FIG. 15 and FIG. 17B).

However, in KOR specific agonist U69593 pretreated membranes, 6β-naltrexol turned into inverse agonist as naloxone did, while 6β-naltrexamide remains neutral (as shown in FIG. 16 and FIG. 17A-17B).

In contrast to naloxone, naltrexone was neutral antagonist in DOR and KOR even after agonist pretreatment (as shown in FIG. 15A-15B and FIG. 16).

These results indicate that different opioid receptors are subject to different regulation upon agonist pretreatment and that antagonists can have different properties at three opioid receptors.

Since MOR is the major receptor in mediating opioid dependence and withdrawal, the inventors further tested these two compounds (6β-naltrexol and 6β-naltrexamide) in permeabilized MOR cells using GTPγS binding assay.

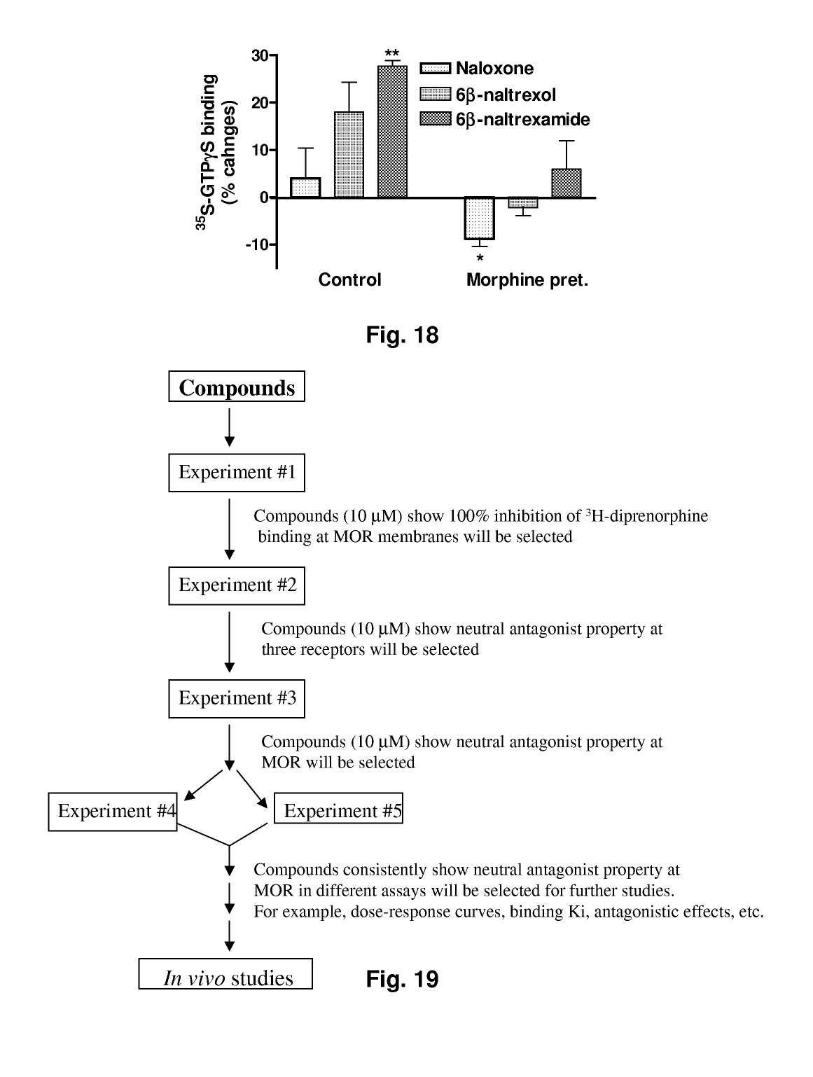

Consistent with the results from membranes GTPγS binding assay, 6β-naltrexol and 6β-naltrexamide showed neutral antagonist property (FIG. 18).

These two compounds were also tested in animals. Since 6β-naltrexol has a neutral antagonist property, 6β-naltrexol precipitated less withdrawal jumping than naloxone. 6β-naltrexamide is a peripheral opioid antagonist; consequently, it caused less diarrhea in opioid pretreated animals. Therefore, neutral antagonists selected by the in vitro screen methods are also neutral antagonists in vivo and precipitate less withdrawal.

Definitions and Abbreviations: GPCR, G protein-coupled receptor; MOR, μ-opioid receptor; DOR, δ-opioid receptor; KOR, κ-opioid receptor; BNTX, 7-benzylidenenaltrexone; ICI 174,864, N,N-diallyl-Tyr-Aib-Aib-Phe-Leu-OH; nor-BNI, nor-binaltorphimine; GNTI, 5′-guanidinyl-17-(cyclopropylmethyl)-6,7-dehydro-4,5α-epoxy-3,14-dihydroxy-6,7-2′3′-indolomorphinan dihydrochloride; U-69593, (+)-(5α,7α,8β)-N-methyl-N-[7-(1-pyrrolidinyl)-1-oxaspiro[4.5]dec-8-yl]benzeneacetamide; DPDPE, [D-Pen2,D-Pen5]-enkephalin; DAMGO, [D-Ala2,N-Me-Phe4,Gly5-ol]-enkephalin; U50,488H, trans-(±)-3,4-dichloro-N-methyl-N-[2-(1-pyrrolidinyl)-cyclohexyl]benzeneacetamide methane-sulfonate hydrate; GTPγS, guanosine 5′-O-(3-thio)triphosphate; HEK, human embryonic kidney; PBS, phosphate-buffered saline; Nal, naloxone; 6β-nal, 6β-naltrexol; 6β-NXM, 6β-naltrexamide; ANOVA, analysis of variance.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

The citation of any reference herein is not an admission that such reference is available as prior art to the instant invention. Any publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.
Becker J A, Wallace A, Garzon A, Ingallinella P, Bianchi E, Cortese R, Simonin F, Kieffer B L, and Pessi A (1999) Ligands for κ-opioid and ORL1 receptors identified from a conformationally constrained peptide combinatorial library. J Biol Chem 274: 27513-27522.
Bilsky E J, Bernstein R N, Wang Z, Sadee W, and Porreca F (1996) Effects of naloxone and D-Phe-Cys-Tyr-D-Trp-Arg-Thr-Pen-Thr-NH₂ and the protein kinase inhibitors H7 and H8 on acute morphine dependence and antinociceptive tolerance in mice. J Pharmacol Exp Ther 277: 484-490.
Brillet K, Kieffer B L, and Massotte D (2003) Enhanced spontaneous activity of the mu opioid receptor by cysteine mutations: characterization of a tool for inverse agonist screening. BMC Pharmacol 3: 14.
Burford N T, Wang D, and Sadee W (2000) G-protein coupling of mu-opioid receptors (OP3): elevated basal signalling activity. Biochem J 348: 531-537.
Chakrabarti S, Oppermann M, and Gintzler A R (2001) Chronic morphine induces the concomitant phosphorylation and altered association of multiple signaling proteins: a novel mechanism for modulating cell signaling. Proc Natl Acad Sci USA 98: 4209-4214.
Chick J, Anton R, Checinski K, Croop R, Drummond DC, Farmer R, Labriola D, Marshall J, Moncrieff J, Morgan M Y, et al. (2000) A multicentre, randomized, double-blind, placebo-controlled trial of naltrexone in the treatment of alcohol dependence or abuse. Alcohol 35: 587-593.
Chiu C T, Ma T, and Ho I K (2006) Methamphetamine-induced behavioral sensitization in mice: alterations in mu-opioid receptor. J Biomed Sci 13: 797-811
Cone E J, Gorodetzky C W, and Yeh S Y (1974) The urinary excretion profile of naltrexone and metabolites in man. Drug Metab Dispos 2: 506-512.
Costa T and Herz A (1989) Antagonists with negative intrinsic activity at delta opioid receptors coupled to GTP-binding proteins. Proc Natl Acad Sci USA 86: 7321-7325.
Davidson D, Palfai T, Bird C, and Swift R (1999) Effects of naltrexone on alcohol self-administration in heavy drinkers. Alcohol Clin Exp Res 23: 195-203.
Dupre D J, Rola-Pleszczynski M, and Stankova J (2004) Inverse agonism: more than reverting constitutively active receptor signaling. Biochem Cell Biol 82: 676-680.
Emmerson P J, McKinzie J H, Surface P L, Suter T M, Mitch C H, and Statnick M A (2004) Na+ modulation, inverse agonism, and anorectic potency of 4-phenylpiperidine opioid antagonists. Eur J Pharmacol 494: 121-130.
Freye E and Levy J (2005) Constitutive opioid receptor activation: a prerequisite mechanism involved in acute opioid withdrawal. Addict Biol 10: 131-137.
Gbahou F, Rouleau A, Morisset S, Parmentier R, Crochet S, Lin J S, Ligneau X, Tardivel-Lacombe J, Stark H, Schunack W, et al. (2003) Protean agonism at histamine H3 receptors in vitro and in vivo. Proc Natl Acad Sci USA 100: 11086-11091.
Gonzalez G, Oliveto A, and Kosten T R (2004) Combating opiate dependence: a comparison among the available pharmacological options. Expert Opin Pharmacother 5: 713-725.
Heinzen E L, Booth R G, and Pollack G M (2005) Neuronal nitric oxide modulates morphine antinociceptive tolerance by enhancing constitutive activity of the muopioid receptor. Biochem Pharmacol 69: 679-688.
Herrick-Davis K, Grinde E, and Teitler M (2000) Inverse agonist activity of atypical antipsychotic drugs at human 5-hydroxytryptamine2C receptors. J Pharmacol Exp Ther 295: 226-232 .
Hollister L E, Johnson K, Boukhabza D, and Gillespie H K (1981) Aversive effects of naltrexone in subjects not dependent on opiates. Drug Alcohol Depend 8: 37-41. Kenakin T (2004) Efficacy as a vector: the relative prevalence and paucity of inverse agonism. Mol Pharmacol 65: 2-11.
Khotib J, Narita M, Suzuki M, Yajima Y, and Suzuki T (2004) Functional interaction among opioid receptor types: up-regulation of mu- and delta-opioid receptor functions after repeated stimulation of kappa-opioid receptors. Neuropharmacology 46: 531-540.
Liu J G and Prather P L (2001) Chronic exposure to μ-opioid agonists produces constitutive activation of μ-opioid receptors in direct proportion to the efficacy of the agonist used for pretreatment. Mol Pharmacol 60: 53-62.
Liu J G and Prather P L (2002) Chronic agonist treatment converts antagonists into inverse agonists at δ-opioid receptors. J Pharmacol Exp Ther 302: 1070-1079.
Liu J G, Ruckle M B, and Prather P L (2001) Constitutively active μ-opioid receptors inhibit adenylyl cyclase activity in intact cells and activate G-proteins differently than the agonist [D-Ala2,N-MePhe4,Gly-ol5]enkephalin. J Biol Chem 276: 37779-37786.
McCaul M E, Wand G S, Rohde C, and Lee S M (2000) Serum 6-beta-naltrexol levels are related to alcohol responses in heavy drinkers. Alcohol Clin Exp Res 24: 1385-1391.
Narita M, Funada M, and Suzuki T (2001) Regulations of opioid dependence by opioid receptor types.
Pharmacol Ther 89: 1-15.
Pineyro G, Azzi M, deLean A, Schiller P W, and Bouvier M (2005) Reciprocal regulation of agonist and inverse agonist signaling efficacy upon short-term treatment of the human ε-opioid receptor with an inverse agonist. Mol Pharmacol 67: 336-348.
Raehal K M, Lowery J J, Bhamidipati C M, Paolino R M, Blair J R, Wang D, Sadee W, and Bilsky E J (2005) In vivo characterization of 6β-naltrexol, an opioid ligand with less inverse agonist activity compared with naltrexone and naloxone in opioid-dependent mice. J Pharmacol Exp Ther 313: 1150-1162.
Rukstalis M R, Stromberg M F, O'Brien C P, and Volpicelli J R (2000) 6-β-Naltrexol reduces alcohol consumption in rats. Alcohol Clin Exp Res 24: 1593-1596.
Sadee W, Wang D, and Bilsky E J (2005) Basal opioid receptor activity, neutral antagonists, and therapeutic opportunities. Life Sci 76: 1427-1437.
Shoblock J R and Maidment N T (2006) Constitutively active mu opioid receptors mediate the enhanced conditioned aversive effect of naloxone in morphine-dependent mice. Neuropsychopharmacology 31: 171-177.
Stromberg M F, Rukstalis M R, Mackler S A, Volpicelli J R, and O'Brien C P (2002) A comparison of the effects of 6-beta naltrexol and naltrexone on the consumption of ethanol or sucrose using a limited-access procedure in rats. Pharmacol Biochem Behav 72: 483-490.
Verebey K, Volavka J, Mule S J, and Resnick R B (1976) Naltrexone: disposition, metabolism, and effects after acute and chronic dosing. Clin Pharmacol Ther 20: 315-328.
Walker E A and Sterious S N (2005) Opioid antagonists differ according to negative intrinsic efficacy in a mouse model of acute dependence. Br J Pharmacol 145: 975-983.
Wang D, Raehal K M, Bilsky E J, and Sadee W (2001) Inverse agonists and neutral antagonists at mu opioid receptor (MOR): possible role of basal receptor signaling in narcotic dependence. J Neurochem 77: 1590-1600.
Wang D, Raehal K M, Lin E T, Lowery J J, Kieffer B L, Bilsky E J, and Sadee W (2004) Basal signaling activity of mu opioid receptor in mouse brain: role in narcotic dependence. J Pharmacol Exp Ther 308: 512-520.
Wang D, Sadee W, and Quillan J M (1999) Calmodulin binding to G protein-coupling domain of opioid receptors. J Biol Chem 274: 22081-22088.
Wang D, Sun X, Bohn L M, and Sadee W (2005) Opioid receptor homo- and heterodimerization in living cells by quantitative bioluminescence resonance energy transfer. Mol Pharmacol 67: 2173-2184.
Wang D, Surratt C K, and Sadee W (2000) Calmodulin regulation of basal and agonist-stimulated G protein coupling by the mu-opioid receptor (OP(3)) in morphine-pretreated cell. J Neurochem 75: 763-771.
Wang Z, Bilsky E J, Porreca F, and Sadee W (1994) Constitutive mu opioid receptor activation as a regulatory mechanism underlying narcotic tolerance and dependence. Life Sci 54: PL339-PL350.
Zaki P A, Keith D E Jr, Thomas J B, Carroll F I, and Evans C J (2001) Agonist-, antagonist-, and inverse agonist-regulated trafficking of the δ-opioid receptor correlates with, but does not require, G protein activation. J Pharmacol Exp Ther 298: 1015-1020.

1. A method for determining whether agonist-pretreatment increases basal activity and/or sensitizes inverse agonist effects at MOR, DOR and/or KOR, comprising,
screening of opioid receptor neutral antagonists and inverse agonists, including
providing cells stably expressing single μ-opioid receptor (MOR), δ-opioid receptor (DOR) or κ-opioid receptor (KOR), or co-expressing MOR/DOR, MOR/KOR or DOR/KOR;
selecting one or more compounds showing inhibition of ³H-diprenorphine binding at MOR membranes;
selecting one or more compounds showing neutral agonist properties at three receptors;
selecting one or more compounds showing neutral agonist properties at MOR; and,
selecting one or more compounds substantially consistently showing neutral antagonist properties at MOR in different assays.

2. The method of claim 1, wherein each compound is tested in three receptors, without or with a narcotic analgesic or receptor specific agonist pretreatment.

3. The method of claim 2, wherein the narcotic analgesic is morphine, and the receptor specific agonist pretreatment is an inverse agonist.

4. The method of claim 3, wherein the receptor specific agonist pretreatment is an inverse agonist comprising β-naloxone.

5. The method of claim 1, wherein cells stably expressing single MOR, DOR or KOR, or co-expressing MOR/DOR, MOR/KOR or DOR/KOR are established by:
transfecting opioid receptors into HEK cells, and selecting single clones;
culturing one or more cloned cells,
pretreating the cells with morphine or subtype specific agonists,
harvesting one or more the cells; using permeabilized cells or cell membranes in a GTPγS, guanosine 5′-O-(3-thio)triphosphate (GTPγS) binding assay with the one or more compounds; and,
incubating the one or more compounds with the cell membranes or permeabilized cells and ³H-diprenorphine.

6. The method of claim 5, wherein the pretreating step comprises pretreating the cells with morphine or subtype specific agonists comprises DAMGO, [D-Ala2,N-Me-Phe4,Gly5-ol]-enkephalin, for MOR.

7. The method of claim 6, wherein the ³⁵S-GTPγS binding assay in membranes uses an assay buffer containing Tris-HCl, KCl, EDTA, MgCl₂, GDP and ³⁵S-GTPγS.

8. The method of claim 5, wherein the pretreating step comprises pretreating the cells with morphine or subtype specific agonists comprises, DPDPE, [D-Pen2,D-Pen5]-enkephalin, for DOR.

9. The method of claim 8, wherein the ³⁵S-GTPγS binding assay in membranes uses an assay buffer containing Tris-HCl, KCl, EDTA, MgCl₂, GDP and ³⁵S-GTPγS.

10. The method of claim 5, wherein the pretreating step comprises pretreating the cells with morphine or subtype specific agonists comprises U69593, (+)-(5αa,7α,8α)-N-methyl-N-[7-(1-pyrrolidinyl)-1-oxaspiro [4.5] dec-8-yl] benzeneacetamide, for KOR.

11. The method of claim 10, wherein the ³⁵S-GTPγS binding assay in membranes uses an assay buffer containing Tris-HCl, NaCl, EDTA, MgCl₂, GDP and ³⁵S-GTPγS.

12. The method of claim 5, wherein in the ³⁵S-GTPγS binding assay in permeabilized cells, the one or more compounds were incubated with the permeabilized cells and ³⁵S-GTPγS.

13. The method of claim 5, wherein in the assay, the cells pretreated with or without morphine or receptor specific agonists, then incubated with the one or more compounds.

14. A method for testing the effects of opioid antagonists on MOR, DOR, KOR and their hetero-dimers before or after different agonists pretreatment, comprising using the method of claim 1.

15. A method for identifying potential side-effects of opioid antagonists, comprising using the method of claim 1.

16. A method for selecting a drug candidates for treatment of conditions where opioid antagonists are indicated, comprising using the method of claim 1.

17. The method as in claim 1, including comparing of receptor activities by classifying a test compound as an agonist, neutral antagonist, or an inverse agonist.

18. A kit comprising an assay for the screening method of claim 1.

19. The kit of claim 18, further comprising instructions for correlating the assay results with the subject's risk for having or developing an adverse withdrawal symptom.

20. The kit of claim 18, further comprising instructions for correlating the assay results with the subject's prognostic outcome for an adverse withdrawal symptom.

21. The kit of claim 18, further comprising instructions for correlating the assay results with the probability of success or failure of a particular drug treatment in the subject.

22. A method for distinguishing the effects of naloxone from those of naltrexone comprising using the method of claim 1.

23. A method for demonstrating different antagonists having distinct pharmacological properties comprising using the method of claim 1.

24. (canceled)

25. The method of claim 1, wherein, for MOR, both potency and efficacy of BNTX, 7-benzylidenenaltrexone, increases, and naloxone and naltrexone turn into inverse agonist after agonist pretreatment.

26. The method of claim 1, wherein, for KOR, agonist pretreatment increases efficacy and potency of nor-BNI, nor-binaltorphimine, and GNTI, 5′-guanidinyl-17-(cyclopropylmethyl)-6,7-dehydro-4,5α-epoxy-3,14-dihydroxy-6,7-2′3′-indolomorphinan dihydrochloride, affects potency.

27. The method of claim 1, wherein, for DOR, a decrease in efficacy of ICI 174,864, N,N-diallyl-Tyr-Aib-Aib-Phe-Leu-OH, increases after DPDPE, [D-Pen2,D-PenS]-enkephalin, pretreatment, an/or the potency of ICI 174,864 increases after morphine pretreatment.

28. A method for developing safer and more effective opioid antagonists targeting a variety of clinical needs, including long-term treatment of addiction, and opioid-induced gastrointestinal dysfunction, comprising using the method of claim 1.

29. An assay for determining the chemosensitivity of a subject to a particular opioid drug, comprising screening a sample from the subject using the method of claim 1.

30. The assay of claim 29, further including detecting a chemosensitivity profile in the subject, and determining whether the subject is experiencing an opioid drug reaction or interaction.

31. The assay of claim 29, further including predicting the effect of a particular opioid drug on the subject.

32. A method for treatment of opioid drug interactions in a subject, comprising screening a sample from the subject using the method of claim 1, and further determining a treatment regime for the patient.
